(19) United States
(12) Patent Application Publication
(10) Pub. No.: US 2017/0064494 A1
(43) Pub. Date: Mar. 2, 2017

(54) SKIN MOISTURE TESTING SYSTEM AND METHOD

(71) Applicant: Shenzhen Hali-Power Industrial Co., Ltd., Shenzhen (CN)

(72) Inventor: Xiaomin ZHU, Shenzhen (CN)

(21) Appl. No.: 15/014,041

(22) Filed: Feb. 3, 2016

(30) Foreign Application Priority Data
Sep. 2, 2015 (CN) 201510556210.3

(51) Int. Cl. H04W 4/00; H04W 4/06; A61B 5/00; H04L 12/26

(52) U.S. Cl. CPC H04W 4/008; H04L 43/045; H04W 4/06; A61B 5/443

(57) ABSTRACT

A skin moisture testing system comprises a Bluetooth MCU control unit, a Bluetooth antenna impedance matching unit, a RC integral and diode rectifier unit, a sinusoidal signal unit, a first contact, a second contact. A skin moisture testing method matches with a lower powered Bluetooth MCU to test a user's skin for a long time, and the tested skin moisture value can be visually displayed on the APP of the terminal device, the testing data can be made to form a graph.

### BACKGROUND

1. Technical Field

The present disclosure generally relates to a testing region, and especially relates to a skin moisture testing system and a method of testing skin moisture value.

2. Description of Related Art

Traditional cosmetics can only provide skin moisture; however, the traditional cosmetics do not know about skin moisture conditions, and cannot provide a scientific and reasonable moisturizing solution to the skin. The present invention aims to let consumers intuitively know his/her own skin condition, and provide the skin moisture in time. We can learn more about our skin, and care for our skin more scientifically and reasonably.

Therefore, a need exists in the industry to overcome the described problems.

### SUMMARY

The disclosure is to offer a skin moisture testing system and a method of testing the skin moisture value.

A skin moisture testing system comprises a Bluetooth MCU control unit, a Bluetooth antenna impedance matching unit including an IC matching circuit and a terminal matching circuit, a RC integral and diode rectifier unit, a sinusoidal signal unit, a first contact, a second contact, the first contact and the second contact in contact with skin of a user; the sinusoidal signal unit transmits a square wave high frequency sinusoidal signal emitted by the Bluetooth MCU control unit to the first contact, the second contact receives a first signal via the Bluetooth MCU control, the first signal is filtered and shaped by the RC integral and diode rectifier unit to a second signal, the Bluetooth MCU control unit samples the second signal to get a plurality of samples, the plurality of samples quantified to get a semaphore value; the Bluetooth antenna impedance matching unit translates the semaphore value into a skin impedance value, the skin impedance value is translated into a skin moisture content value corresponding to the skin impedance value, the Bluetooth antenna impedance matching unit matches with the Bluetooth MCU control unit, and partly matching with an on board antenna of the Bluetooth antenna.

Preferably, the high frequency signal is a 4 KHz±500 Hz sinusoidal signal.

Preferably, a ninth terminal of the Bluetooth MCU control unit is connected with the RC integral and diode rectifier unit, and is used to detect integral values.

Preferably, a fifth terminal of the Bluetooth MCU control unit is connected with a resistor and a light emitting diode, and is used to control the light emitting diode.

Preferably, a fifteenth terminal of the Bluetooth MCU control unit emits the 4 KHz±500 Hz sinusoidal signal and transmits the 4 KHz±500 Hz sinusoidal signal to the first contact.

Preferably, a thirty first terminal of the Bluetooth MCU control unit and a thirty second terminal of the Bluetooth MCU control unit are connected with the Bluetooth antenna impedance matching unit.

Preferably, a circuit of the Bluetooth antenna impedance matching unit includes a first coil, a second coil, a third coil, a fourth coil, a third capacitor, a fourth capacitor, a fifth capacitor, a sixth capacitor, a fourteenth capacitor, a sixteenth capacitor, and an antenna; the antenna, the sixteenth capacitor, the fourteenth capacitor, the fifth capacitor, the first coil, the second coil, the third capacitor are cascaded with each other in sequence, and then grounded, a lead between the first coil and the fifth capacitor is connected with the thirty second terminal of the Bluetooth MCU control unit, another lead between the first coil and the second coil is connected with the thirty first terminal of the Bluetooth MCU control unit, a lead between the second coil and the third capacitor is connected with a thirtieth terminal of the Bluetooth MCU control unit, the fourth capacitor is connected with the fifth capacitor and the fourteenth capacitor, and then grounded, the fourth capacitor is located between the fifth capacitor and the fourteenth capacitor, the fourteenth capacitor and the sixteenth capacitor are connected with the sixth capacitor and the second coil in parallel, and then grounded, the fourth coil is connected with the sixteenth capacitor and the antenna, and then grounded, the fourth coil is located between the sixteenth capacitor and the antenna.

Preferably, the Bluetooth MCU control unit is a chip having 48 foot, a first terminal of the chip is connected with a power VCC, and the first terminal of the chip is grounded through a seventh capacitor; a seventh terminal of the chip is connected with a switch; a twelfth terminal of the chip is connected with the power VCC, and the twelfth terminal of the chip is grounded through a eleven capacitor; a thirteen terminal of the chip is grounded; a twenty fourth terminal is grounded by a first resistor, a twenty third terminal is connected with a D point, the twenty fourth terminal is connected with a C point, a G point is grounded, and the D point, the C point and the G point are burning points; a twenty ninth terminal is grounded through a tenth capacitor, a thirty third terminal and a thirty fourth terminal are both grounded, a thirty fifth terminal and a thirty sixth terminal are both connected with the power VCC, the thirty fifth terminal and the thirty sixth terminal are grounded through a ninth capacitor, a thirty seventh terminal is grounded through a first capacitor, a thirty eighth terminal is grounded through the second capacitor, a crystal oscillator is located between the thirty seventh terminal and the thirty eighth terminal, and the crystal oscillator is connected with the thirty eighth terminal and the thirty eighth terminal, a thirty ninth terminal is grounded through a eighth capacitor.

A method of testing skin moisture value using a skin moisture system comprising a Bluetooth MCU control unit, including: an application installed in a terminal having a terminal Bluetooth antenna; the Bluetooth MCU control unit transmitting a broadcast; the terminal device recognizing the broadcast after the terminal Bluetooth antenna matching with the Bluetooth MCU control unit; the two contacts contacting a skin of a user and the skin moisture testing system testing the skin moisture value of the user; and a tested skin moisture value calculated by the Bluetooth MCU control unit and transmitted to the terminal device to display to the user.

Preferably, the Bluetooth MCU control unit transmits the broadcast by pressing the power button.

Preferably, before the terminal Bluetooth antenna is matched and connected with the Bluetooth MCU control unit, the user selects to boot the terminal Bluetooth antenna on the terminal device, then selects a corresponding service set identifier of the broadcast on the application of the terminal device.

Preferably, the skin moisture testing system further comprises a Bluetooth antenna impedance matching unit, a RC integral and diode rectifier unit, a sinusoidal signal unit, a first contact, a second contact, the first contact and the second contact in contact with skin of the user.

Preferably, the high frequency signal is a 4 KHz±500 Hz sinusoidal signal.

Preferably, a ninth terminal of the Bluetooth MCU control unit is connected with the RC integral and diode rectifier unit, and is used to detect integral values.

Preferably, a fifth terminal of the Bluetooth MCU control unit is connected with a resistor and a light emitting diode, and is used to control the light emitting diode.

Preferably, a fifteenth terminal of the Bluetooth MCU control unit emits the 4 KHz±500 Hz sinusoidal signal and transmits the 4 KHz±500 Hz sinusoidal signal to the first contact.

Preferably, a thirty first terminal of the Bluetooth MCU control unit and a thirty second terminal of the Bluetooth MCU control unit are connected with the Bluetooth antenna impedance matching unit.

Preferably, a circuit of the Bluetooth antenna impedance matching unit includes a first coil, a second coil, a third coil, a fourth coil, a third capacitor, a fourth capacitor, a fifth capacitor, a sixth capacitor, a fourteenth capacitor, a sixteenth capacitor, and an antenna; the antenna, the sixteenth capacitor, the fourteenth capacitor, the fifth capacitor, the first coil, the second coil, the third capacitor are cascaded with each other in sequence, and then grounded, a lead between the first coil and the fifth capacitor is connected with the thirty second terminal of the Bluetooth MCU control unit, another lead between the first coil and the second coil is connected with the thirty first terminal of the Bluetooth MCU control unit, a lead between the second coil and the third capacitor is connected with a thirtieth terminal of the Bluetooth MCU control unit, the fourth capacitor is connected with the fifth capacitor and the fourteenth capacitor, and then grounded, the fourth capacitor is located between the fifth capacitor and the fourteenth capacitor, the fourteenth capacitor and the sixteenth capacitor are connected with the sixth capacitor and the second coil in parallel, and then grounded, the fourth coil is connected with the sixteenth capacitor and the antenna, and then grounded, the fourth coil is located between the sixteenth capacitor and the antenna.

Preferably, the Bluetooth MCU control unit has a power button, and is used to do AD testing, LED indication controlling, antenna impedance matching, and the Bluetooth MCU control unit is a chip having 48 terminals, a first terminal of the chip is connected with a power VCC, and the first terminal of the chip is grounded through a seventh capacitor; a seventh terminal of the chip is connected with a switch; a twelfth terminal of the chip is connected with the power VCC, and the twelfth terminal of the chip is grounded through a eleven capacitor; a thirteen terminal of the chip is grounded; a twenty fourth terminal is grounded by a first resistor, a twenty third terminal is connected with a D point, the twenty fourth terminal is connected with a C point, a G point is grounded, and the D point, the C point and the G point are burning points; a twenty ninth terminal is grounded through a tenth capacitor, a thirty third terminal and a thirty fourth terminal are both grounded, a thirty fifth terminal and a thirty sixth terminal are both connected with the power VCC, the thirty fifth terminal and the thirty sixth terminal are grounded through a ninth capacitor, a thirty seventh terminal is grounded through a first capacitor, a thirty eighth terminal is grounded through the second capacitor, a crystal oscillator is located between the thirty seventh terminal and the thirty eighth terminal, and the crystal oscillator is connected with the thirty eighth terminal and the thirty eighth terminal, a thirty ninth terminal is grounded through a eighth capacitor.

Compared to the traditional skin moisture testing system, the skin moisture testing method of the present invention is motivated by a pulse signal, the skin moisture testing method can effectively and accurately test skin moisture value. The skin moisture testing method also matches with a lower powered Bluetooth MCU to test user's skin for a long time, then the tested skin moisture value can be visually displayed on the APP of the terminal device, the testing data can be made to form a graph, such that the skin moisture testing method can test comprehensive skin nutrition moisture percentage under the basis of an guarantee of no damaging to the skin, and the skin moisture testing method can guide the care of the skin.

### BRIEF DESCRIPTION OF THE DRAWINGS

Many aspects of the present embodiments can be better understood with reference to the following drawings. The components in the drawings are not necessarily drawn to scale, the emphasis instead being placed upon clearly illustrating the principles of the present embodiments. Moreover, in the drawings, all the views are schematic, and like reference numerals designate corresponding parts throughout the several views.

FIG. 1 is a schematic diagram of a skin moisture testing system;

FIG. 2 is an isometric view of a circuit structure;

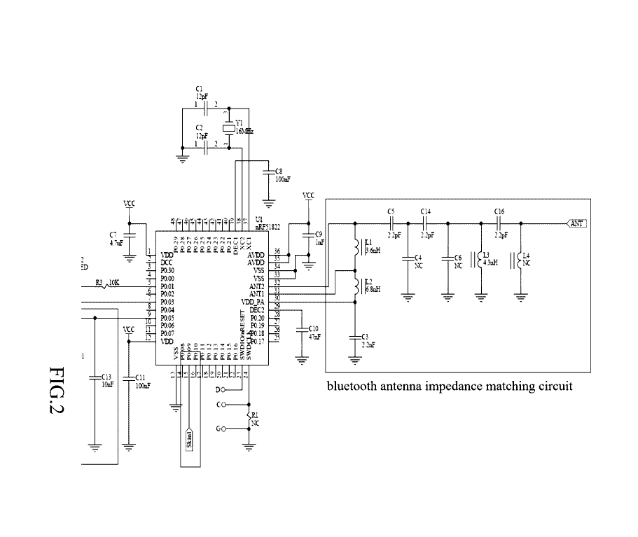

FIG. 3 is an isometric view of a Bluetooth MCU control unit;

FIG. 4 is an isometric view of a Bluetooth antenna impedance matching unit;

FIG. 5 is a structure diagram of a sinusoidal signal unit;

FIG. 6 is a structure diagram of a RC integral and diode rectifier unit;

FIG. 7 is a flow chart of a method of testing a skin moisture value based on the skin moisture testing system.

### DETAILED DESCRIPTION

The disclosure is illustrated by way of example and not by way of limitation in the figures of the accompanying drawings, in which like reference numerals indicate similar elements. It should be noted that references to “an” or “one” embodiment in this disclosure are not necessarily to the same embodiment, and such references can mean “at least one” embodiment.

A skin moisture testing system includes a Bluetooth MCU control unit 1 (Micro controller Unit, MCU), a Bluetooth antenna impedance matching unit 2, an AD detecting unit 3 which can also be defined as a RC integral and diode rectifier unit which includes a RC integral circuit 31 and a diode rectification 33, a sinusoidal signal unit 17, a first contact 4 which is presented as Skin 1, a second contact 5 which is presented as Skin 2. The Bluetooth MCU control unit 1 has the following functions, such as AD testing 11, LED indication controlling 13 (light emitting diode, LED), antenna impedance matching 19, the Bluetooth MCU control unit 1 also has a power button 15, the antenna impedance matching unit 2 includes an IC matching circuit 21 and a terminal matching circuit 23, the sinusoidal signal unit 17 can transmit the sinusoidal signal which can be a square wave of a high frequency sinusoidal signal, the sinusoidal signal is emitted by the Bluetooth MCU control unit 1 and transmitted to the first contact 4, after a current passes through the skin, the current can return back to the Bluetooth MCU control unit 1 through the second contact 5, at this time the sinusoidal signal has already been changed, the changed sinusoidal signal can be signal filtered and shaping treated by the AD detecting unit 3, the Bluetooth MCU control unit 1 starts sampling to get a plurality of samples, and then the samples are quantified to get a semaphore value, then the Bluetooth antenna impedance matching unit 2 can translate the semaphore value into a skin impedance value, a transformation equation is established through a data fitting analysis, finally the skin impedance value is translate into a skin moisture content value corresponding to the skin impedance value, the Bluetooth antenna impedance matching unit 2 is responsible for matching with the Bluetooth MCU control unit 1, and partly matching with an on board antenna, such that the skin moisture testing system of the present invention can remotely and reliably transport the data.

The high frequency signal can be 4 KHz±500 Hz sinusoidal signal, such as 3700 Hz, 3800 Hz, 3900 Hz, 4000 Hz, 4300 Hz, and 4400 Hz.

A ninth terminal of the Bluetooth MCU control unit 1 is connected with the RC integral and diode rectifier unit, and can be used to test integral values.

A fifth terminal of the Bluetooth MCU control unit 1 is connected with a resistor R8 and a light emitting diode (LED), and can be used to control a state of the light emitting diode.

A fifteenth terminal of the Bluetooth MCU control unit 1 emits 4 KHz±500 Hz sinusoidal signal and transmits the 4 KHz±500 Hz sinusoidal signal to the first contact 4.

A thirty first terminal of the Bluetooth MCU control unit 1 and a thirty second terminal of the Bluetooth MCU control unit 1 are connected with the Bluetooth antenna impedance matching unit 2.

A circuit of the Bluetooth antenna impedance matching unit 2 includes a first coil L1, a second coil L2, a third coil L3, a fourth coil L4, a third capacitor C3, a fourth capacitor C4, a fifth capacitor C5, a sixth capacitor C6, a fourteenth capacitor C14, a sixteenth capacitor C16, and an antenna; the antenna, the sixteenth capacitor C16, the fourteenth capacitor C14, the fifth capacitor C5, the first coil L1, the second coil L2, the third capacitor C3 are cascaded with each other in sequence, and then grounded, a lead between the first coil L1 and the fifth capacitor C5 is connected with the thirty second terminal of the Bluetooth MCU control unit 1, another lead between the first coil L1 and the second coil L2 is connected with the thirty first terminal of the Bluetooth MCU control unit 1, a lead between the second coil L2 and the third capacitor C3 is connected with a thirtieth terminal of the Bluetooth MCU control unit 1, the fourth capacitor C4 is connected with the fifth capacitor C5 and the fourteenth capacitor C14, and then grounded, the fourth capacitor C4 is located between the fifth capacitor C5 and the fourteenth capacitor C14, the fourteenth capacitor C14 and the sixteenth capacitor C16 are connected with the sixth capacitor C6 and the second coil L3 in parallel, and then grounded, the fourth coil L4 is connected with the sixteenth capacitor C16 and the antenna, and then grounded, the fourth coil L4 is located between the sixteenth capacitor C16 and the antenna.

In at least one exemplary embodiment, the Bluetooth MCU control unit 1 can be a chip which can have forty eight terminals, a first terminal of the chip is connected with a power VCC 6 (Volt Current Condenser, VCC), and the first terminal of the chip is grounded through a seventh capacitor C7; a seventh terminal of the chip is connected with a switch (not shown); a twelfth terminal of the chip is connected with the power VCC 6, and the twelfth terminal of the chip is grounded through a eleven capacitor C11; a thirteen terminal of the chip is grounded; a twenty fourth terminal is grounded by a first resistor R1, a twenty third terminal is connected with a D point, the twenty fourth terminal is connected with a C point, a G point is grounded, and the D point, the C point and the G point are burning points; a twenty ninth terminal is grounded through a tenth capacitor C10, a thirty third terminal and a thirty fourth terminal are both grounded, a thirty fifth terminal and a thirty sixth terminal are both connected with the power VCC 6, the thirty fifth terminal and the thirty sixth terminal are grounded through a ninth capacitor C9, a thirty seventh terminal is grounded through a first capacitor C1, a thirty eighth terminal is grounded through the second capacitor C2, a crystal oscillator Y1 is located between the thirty seventh terminal and the thirty eighth terminal, and the crystal oscillator Y1 is connected with the thirty seventh terminal and the thirty eighth terminal, a thirty ninth terminal is grounded through a eighth capacitor C8.

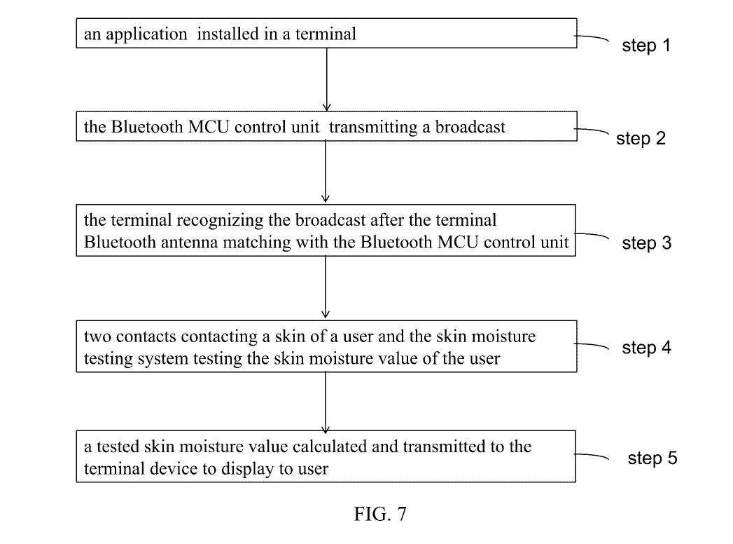

Referring to FIG. 7, FIG. 7 is a flow chart of a method of testing a skin moisture value based on the skin moisture testing system. The method of testing the skin moisture value based on the skin moisture testing system, the method comprising: step 1: an application 51 installed in an terminal 5 having a Bluetooth antenna 53, the terminal 5 being a mobile phone or a tablet personal computer; step 2: the Bluetooth MCU control unit 1 transmitting a broadcast by pressing a power button 15; step 3: the terminal device 5 recognizing the broadcast after the terminal Bluetooth antenna 53 matching with the Bluetooth MCU control unit 1; step 4: the two contacts 4, 5 contacting a skin of a user and the skin moisture testing system testing the skin moisture value of the user; and step 5: a tested skin moisture value calculated by the Bluetooth MCU control unit 1 and transmitted to the terminal device 5 to display to the user.

It is to be understood that, when the Bluetooth MCU control unit 1 sends out the broadcast, the user can select to boot the Bluetooth antenna 53 on the terminal 5, then the user can select a corresponding service set identifier (SSID) of the broadcast on an APP interface of the terminal 5, after the terminal 5 are matched and connected with the skin moisture testing system, two contacts 4, 5 can be contacted with the skin.

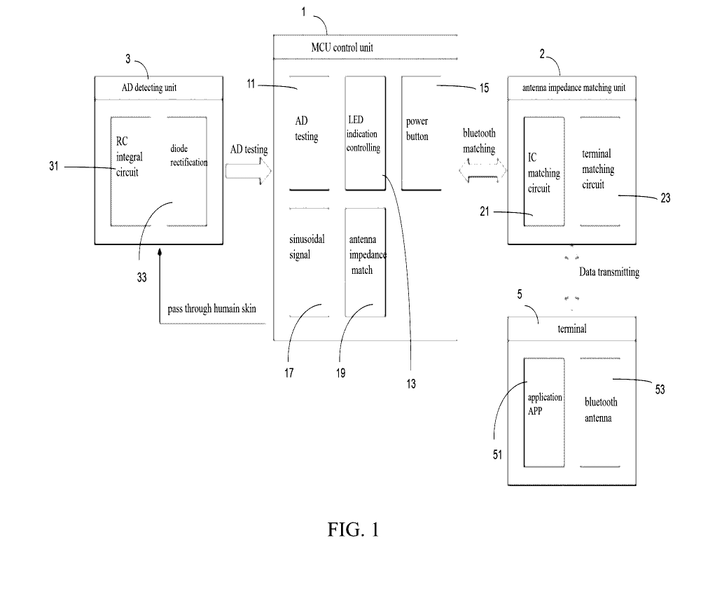

Referring to FIG. 1, FIG. 1 is a schematic diagram of the skin moisture testing system, the skin moisture testing system of the present invention includes the Bluetooth MCU control unit 1, the Bluetooth antenna impedance matching unit 2, the RC integral and diode rectifier unit which can also be defined as the AD detecting unit 3 and the sinusoidal signal unit 17.

The skin moisture testing system can be connected with the terminal 5 and send data to the terminal 5 through the Bluetooth. The terminal 5 can be a phone, a tablet personal computer and so on. The skin moisture testing system can test percentage of comprehensive nutrient water in the skin based on an assurance of not damaging the skin, and a skin care can be guided by a skin moisture testing product using the skin moisture testing system. Testing parts of the skin can include head, face, eye socket, neck, and so on. Firstly, the application 51 can be installed on the terminal 5, and the application 51 can be a MiLi Pure application. The Bluetooth MCU control unit 1 transmits a broadcast, and the terminal device recognizes the broadcast after the terminal Bluetooth matching with the Bluetooth MCU control unit 1. The two contacts 4, 5 contacts the skin of a user and the skin moisture testing system tests the skin moisture value of the user. A tested skin moisture value is calculated by the Bluetooth MCU control unit 1 and is transmitted to the terminal 5 to display to the user.

The square wave of the high frequency sinusoidal signal can be applied on the first contact 4 which is contacted with the skin, the high frequency sinusoidal signal can be 4 KHz±500 Hz sinusoidal signal, a weak small current passes through the skin and then return back through the second contact 5, after the sinusoidal signal is signal filtered, and shaping treated, the Bluetooth MCU control unit 1 starts sampling to get a plurality of samples, the samples are quantified to get a semaphore value, the semaphore value can be transformed into a corresponding skin impedance value, a transformation equation can be established through a data fitting analysis, finally the skin impedance value is translate into skin moisture content value corresponding to the skin impedance value. Tested skin moisture value can be calculated by the Bluetooth MCU algorithm, and then be sent to the application 51 of the terminal 5, such that user can observe the value visually.

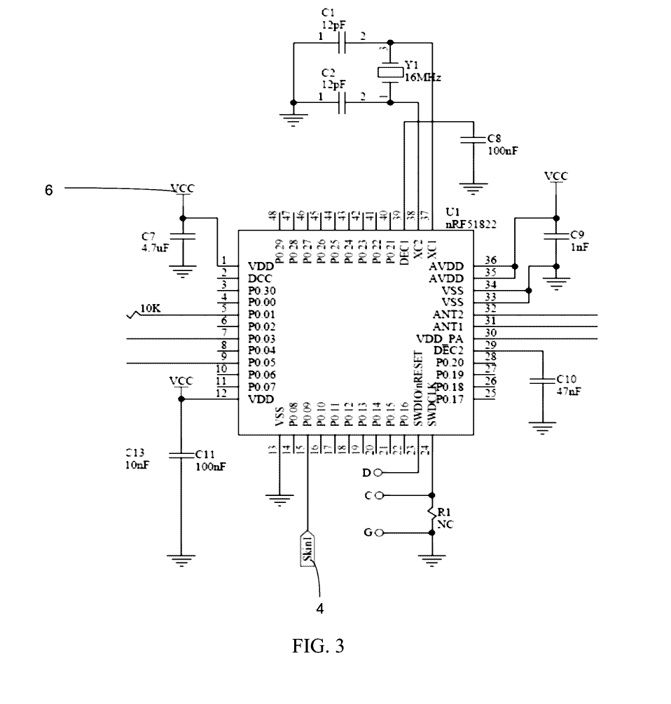

Referring to FIG. 3, FIG. 3 is an isometric view of the Bluetooth MCU control unit 1; the Bluetooth MCU control unit 1 is responsible for AD sampling testing, sending and receiving the data signal, logic control signal, and sending high frequency sinusoidal signal. The ninth terminal is connected with the testing integral value. The fifth terminal is connected with the resistor R8 and the light emitting diode to control a state of the light emitting diode. The fifteenth terminal sends out 4 KHz sinusoidal signal to the first contract 4. The thirty first terminal and the thirty second terminal are connected with the Bluetooth antenna impedance matching unit 2.

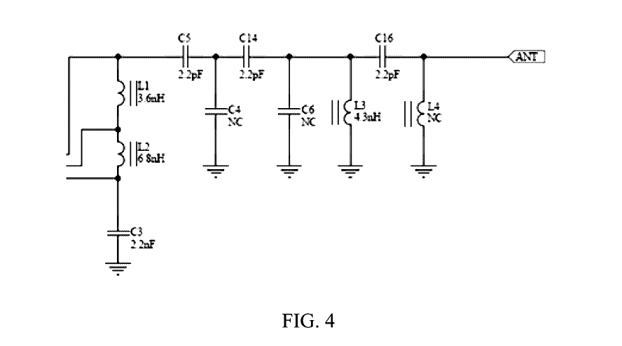

Referring to FIG. 4, FIG. 4 is an isometric view of the Bluetooth antenna impedance matching unit 2, the Bluetooth antenna impedance matching unit 2 is responsible for matching with the Bluetooth MCU control unit 1, and partly matching with the on board antenna, such that the skin moisture testing product using the skin moisture testing system can transmit the data to the terminal 5 reliably and remotely.

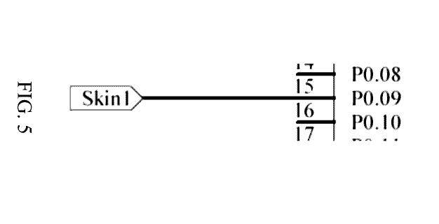

Referring to FIG. 5, FIG. 5 is a structure diagram of the sinusoidal signal unit, the sinusoidal signal unit 17 is responsible for transmitting 4 KHZ sinusoidal signal emitted by the MCU control unit 1 to the first contact 4.

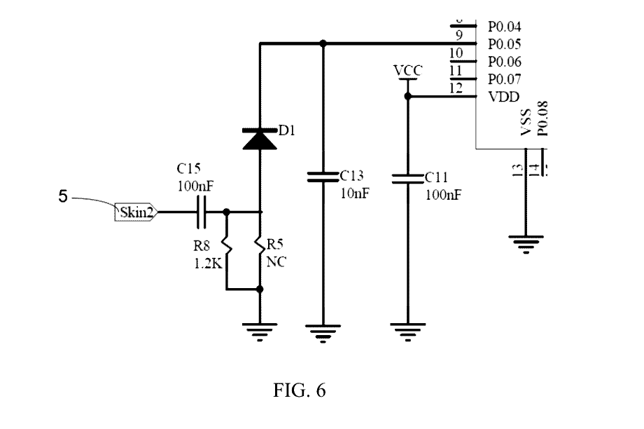

Referring to FIG. 6, FIG. 6 is a structure diagram of the RC integral and diode rectifier unit, a sinusoidal signal emitted by the first contact 4 can pass through the skin, and return to the RC integral and diode rectifier unit through the second contract 5 of the skin moisture testing product using the skin moisture testing system, at this time the sinusoidal signal has already changed, the changed sinusoidal signal is integrated, filtered and shaping treated through the RC circuit 31 which is formed by the fifteenth capacitor C15 and the resistor R8, then the changed sinusoidal signal is returned back to Bluetooth MCU control unit 1, and the Bluetooth MCU control unit 1 starts AD sampling to get a plurality of samples, the samples are quantified to get a semaphore value, the semaphore value is transformed into corresponding skin impedance value. Then the skin impedance value is sent to the terminal device.

Although the features and elements of the present disclosure are described as embodiments in particular combinations, each feature or element can be used alone or in other various combinations within the principles of the present disclosure to the full extent indicated by the broad general meaning of the terms in which the appended claims are expressed.

What is claimed is:

1. A skin moisture testing system, comprising,
a Bluetooth MCU control unit, a Bluetooth antenna impedance matching unit including an IC matching circuit and a terminal matching circuit, a RC integral and diode rectifier unit, a sinusoidal signal unit, a first contact, a second contact, the first contact and the second contact in contact with skin of a user;
the sinusoidal signal unit transmitting a square wave high frequency sinusoidal signal emitted by the Bluetooth MCU control unit to the first contact, the second contact receiving a first signal via the Bluetooth MCU control, the first signal filtered and shaped by the RC integral and diode rectifier unit to a second signal, the Bluetooth MCU control unit sampling the second signal to get a plurality of samples, the plurality of samples quantified to get a semaphore value;
the Bluetooth antenna impedance matching unit translating the semaphore value into a skin impedance value, the skin impedance value is translated into a skin moisture content value corresponding to the skin impedance value, the Bluetooth antenna impedance matching unit matching with the Bluetooth MCU control unit, and partly matching with an on board antenna of the Bluetooth antenna.

2. The skin moisture testing system of claim 1, wherein the high frequency signal is a 4 KHz 500 Hz sinusoidal signal.

3. The skin moisture testing system of claim 1, wherein a ninth terminal of the Bluetooth MCU control unit is connected with the RC integral and diode rectifier unit, and is used to detect integral values.

4. The skin moisture testing system of claim 1, wherein a fifth terminal of the Bluetooth MCU control unit is connected with a resistor and a light emitting diode, and is used to control the light emitting diode.

5. The skin moisture testing system of claim 2, wherein a fifteenth terminal of the Bluetooth MCU control unit emits the 4 KHz±500 Hz sinusoidal signal and transmits the 4 KHz±500 Hz sinusoidal signal to the first contact.

6. The skin moisture testing system of claim 1, wherein a thirty first terminal of the Bluetooth MCU control unit and a thirty second terminal of the Bluetooth MCU control unit are connected with the Bluetooth antenna impedance matching unit.

7. The skin moisture testing system of claim 6, wherein a circuit of the Bluetooth antenna impedance matching unit includes a first coil, a second coil, a third coil, a fourth coil, a third capacitor, a fourth capacitor, a fifth capacitor, a sixth capacitor, a fourteenth capacitor, a sixteenth capacitor, and an antenna; the antenna, the sixteenth capacitor, the fourteenth capacitor, the fifth capacitor, the first coil, the second coil, the third capacitor are cascaded with each other in sequence, and then grounded, a lead between the first coil and the fifth capacitor is connected with the thirty second terminal of the Bluetooth MCU control unit, another lead between the first coil and the second coil is connected with the thirty first terminal of the Bluetooth MCU control unit, a lead between the second coil and the third capacitor is connected with a thirtieth terminal of the Bluetooth MCU control unit, the fourth capacitor is connected with the fifth capacitor and the fourteenth capacitor, and then grounded, the fourth capacitor is located between the fifth capacitor and the fourteenth capacitor, the fourteenth capacitor and the sixteenth capacitor are connected with the sixth capacitor and the second coil in parallel, and then grounded, the fourth coil is connected with the sixteenth capacitor and the antenna, and then grounded, the fourth coil is located between the sixteenth capacitor and the antenna.

8. The skin moisture testing system of claim 1, wherein the Bluetooth MCU control unit is a chip having 48 foot, a first terminal of the chip is connected with a power VCC, and the first terminal of the chip is grounded through a seventh capacitor; a seventh terminal of the chip is connected with a switch; a twelfth terminal of the chip is connected with the power VCC, and the twelfth terminal of the chip is grounded through a eleven capacitor; a thirteen terminal of the chip is grounded; a twenty fourth terminal is grounded by a first resistor, a twenty third terminal is connected with a D point, the twenty fourth terminal is connected with a C point, a G point is grounded, and the D point, the C point and the G point are burning points; a twenty ninth terminal is grounded through a tenth capacitor, a thirty third terminal and a thirty fourth terminal are both grounded, a thirty fifth terminal and a thirty sixth terminal are both connected with the power VCC, the thirty fifth terminal and the thirty sixth terminal are grounded through a ninth capacitor, a thirty seventh terminal is grounded through a first capacitor, a thirty eighth terminal is grounded through the second capacitor, a crystal oscillator is located between the thirty seventh terminal and the thirty eighth terminal, and the crystal oscillator is connected with the thirty eighth terminal and the thirty eighth terminal, a thirty ninth terminal is grounded through a eighth capacitor.

9. A method of testing skin moisture value using a skin moisture system comprising a Bluetooth MCU control unit, including:
an application installed in a terminal having a terminal Bluetooth antenna;
the Bluetooth MCU control unit transmitting a broadcast;
the terminal device recognizing the broadcast after the terminal Bluetooth antenna matching with the Bluetooth MCU control unit;
the two contacts contacting a skin of a user and the skin moisture testing system testing the skin moisture value of the user;
and a tested skin moisture value calculated by the Bluetooth MCU control unit and transmitted to the terminal device to display to the user.

10. The method of claim 9, wherein the Bluetooth MCU control unit transmits the broadcast by pressing the power button.

11. The method of claim 9, wherein before the terminal Bluetooth antenna is matched and connected with the Bluetooth MCU control unit, the user selects to boot the terminal Bluetooth antenna on the terminal device, then selects a corresponding service set identifier of the broadcast on the application of the terminal device.

12. The method of claim 9, wherein the skin moisture testing system further comprises a Bluetooth antenna impedance matching unit, a RC integral and diode rectifier unit, a sinusoidal signal unit, a first contact, a second contact, the first contact and the second contact in contact with skin of the user.

13. The method of claim 12, wherein the high frequency signal is a 4 KHz±500 Hz sinusoidal signal.

14. The method of claim 12, wherein a ninth terminal of the Bluetooth MCU control unit is connected with the RC integral and diode rectifier unit, and is used to detect integral values.

15. The method of claim 12, wherein a fifth terminal of the Bluetooth MCU control unit is connected with a resistor and a light emitting diode, and is used to control the light emitting diode.

16. The method of claim 12, wherein a fifteenth terminal of the Bluetooth MCU control unit emits the 4 KHz±500 Hz sinusoidal signal and transmits the 4 KHz±500 Hz sinusoidal signal to the first contact.

17. The method of claim 12, wherein a thirty first terminal of the Bluetooth MCU control unit and a thirty second terminal of the Bluetooth MCU control unit are connected with the Bluetooth antenna impedance matching unit.

18. The method of claim 17, wherein a circuit of the Bluetooth antenna impedance matching unit includes a first coil, a second coil, a third coil, a fourth coil, a third capacitor, a fourth capacitor, a fifth capacitor, a sixth capacitor, a fourteenth capacitor, a sixteenth capacitor, and an antenna; the antenna, the sixteenth capacitor, the fourteenth capacitor, the fifth capacitor, the first coil, the second coil, the third capacitor are cascaded with each other in sequence, and then grounded, a lead between the first coil and the fifth capacitor is connected with the thirty second terminal of the Bluetooth MCU control unit, another lead between the first coil and the second coil is connected with the thirty first terminal of the Bluetooth MCU control unit, a lead between the second coil and the third capacitor is connected with a thirtieth terminal of the Bluetooth MCU control unit, the fourth capacitor is connected with the fifth capacitor and the fourteenth capacitor, and then grounded, the fourth capacitor is located between the fifth capacitor and the fourteenth capacitor, the fourteenth capacitor and the sixteenth capacitor are connected with the sixth capacitor and the second coil in parallel, and then grounded, the fourth coil is connected with the sixteenth capacitor and the antenna, and then grounded, the fourth coil is located between the sixteenth capacitor and the antenna.

19. The method of claim 12, wherein the Bluetooth MCU control unit has a power button, and is used to do AD testing, LED indication controlling, antenna impedance matching, and the Bluetooth MCU control unit is a chip having 48 terminals, a first terminal of the chip is connected with a power VCC, and the first terminal of the chip is grounded through a seventh capacitor; a seventh terminal of the chip is connected with a switch; a twelfth terminal of the chip is connected with the power VCC, and the twelfth terminal of the chip is grounded through a eleven capacitor; a thirteen terminal of the chip is grounded; a twenty fourth terminal is grounded by a first resistor, a twenty third terminal is connected with a D point, the twenty fourth terminal is connected with a C point, a G point is grounded, and the D point, the C point and the G point are burning points; a twenty ninth terminal is grounded through a tenth capacitor, a thirty third terminal and a thirty fourth terminal are both grounded, a thirty fifth terminal and a thirty sixth terminal are both connected with the power VCC, the thirty fifth terminal and the thirty sixth terminal are grounded through a ninth capacitor, a thirty seventh terminal is grounded through a first capacitor, a thirty eighth terminal is grounded through the second capacitor, a crystal oscillator is located between the thirty seventh terminal and the thirty eighth terminal, and the crystal oscillator is connected with the thirty eighth terminal and the thirty eighth terminal, a thirty ninth terminal is grounded through a eighth capacitor.
